(19) United States
(12) Patent Application Publication
(10) Pub. No.: US 2023/0365873 A1
(43) Pub. Date: Nov. 16, 2023

(54) NITROGEN-FREE HYDROGEN SULPHIDE AND MERCAPTANS SCAVENGERS

(71) Applicant: TOTALENERGIES ONETECH, Courbevoie (FR)

(72) Inventors: Frédéric TORT, GIVORS (FR); Hemant Surendra MONDKAR, NAVI MUMBAI (IN); Jayachandran PERUMALSAMY, NAVI MUMBAI (IN)

(73) Assignee: TOTALENERGIES ONETECH, Courbevoie (FR)

(21) Appl. No.: 18/026,196

(22) PCT Filed: Sep. 15, 2021
(86) PCT No.: PCT/EP2021/075341

§ 371 Date: Mar. 14, 2023

(30) Foreign Application Priority Data
Sep. 18, 2020 (EP) 20306065.2

(51) Int. Cl. C10G 29/22; C07C 43/178; C07C 39/11; C02F 1/68

(52) U.S. Cl. CPC C10G 29/22; C07C 43/1783; C07C 39/11; C02F 1/683; C10G 2300/202; C10G 2300/80; C02F 2101/101

(57) ABSTRACT

In a reaction product of at least one phenol compound with aldehyde(s), the phenol compound(s) reply to formula (II): wherein-R represents a hydrogen atom or a hydrocarbyl group comprising from 1 to 30 carbon atoms, -x is 1 or 2, -R¹ is a linear alkyl or alkenyl group having 15 carbon atoms if x is 1, -R¹ is hydrogen if x is 2, -R² represents a hydrogen atom or a hydrocarbyl group comprising from 1 to 30 carbon atoms, and its use as a hydrogen sulphide and/or mercaptans scavenger in hydrocarbon compositions and in water-based compositions such as water-based drilling fluids.

### TECHNICAL FIELD

The present invention pertains to novel nitrogen free hydrogen sulphide and mercaptans scavengers and to compositions containing the scavengers. The present invention also pertains to the use of the scavengers for scavenging hydrogen sulphide and mercaptans in hydrocarbon containing compositions and in water-containing compositions. The present invention also relates to a method for scavenging hydrogen sulphide and/or mercaptans comprising contacting a media such as crude oils, fuels or natural gas or drilling fluids with the scavenging composition of the invention.

### BACKGROUND OF THE INVENTION

Hydrogen sulphide is a colourless and fairly toxic, flammable and corrosive gas which also has a characteristic odour at a very low concentration. Hydrogen sulphide dissolves in hydrocarbon and water streams and is also found in the vapour phase above these streams and in natural gas. In drilling subterranean wells, notably those for oil or gas, hydrogen suphide can be present in substantial amounts. The drilling fluid may drive the hydrogen sulphide to the surface.

The hydrogen sulphide emissions can therefore be a nuisance to workers operating in the production, transport, processing and storage of oil products such as crude oil, asphalt, Liquid Petroleum Gas, Heavy Fuel Oil, gasoline, kerosene, and diesel fuel. Hydrogen sulphide may also react with hydrocarbon components present in fuel. It would therefore be desirable for the workers' comfort and safety to reduce or even eliminate the hydrogen sulphide emissions during the manipulation of said products.

Legislations have been in place for years, imposing strict regulations on hydrogen sulphide levels of hydrocarbon containing streams in pipelines, in storage and in shipping containers. A variety of chemical scavengers are available to reduce both the concentration and corresponding hazard of hydrogen sulphide in produced gas, crude oil and refined products. The three major classes of hydrogen sulphide scavengers used for the above-mentioned products are water soluble scavengers, oil soluble scavengers and metal-based scavengers.

Among water soluble scavengers, triazines have recently become a more common chemical scavenger used for treating hydrogen sulphide from hydrocarbon containing streams. However, drawbacks linked to the use of triazines can be reported. Triazines, e.g. N-alkyl-triazines, have a nitrogen content between 18-33 mol % which may cause concerns related to residual amines. Most widely used commercial N-alkyl-triazines are hexahydro-1,3,5-trimethyl-1,3,5-triazine (MMA-Triazine) and hexahydro-1,3,5-tris(hydroxyethyl)-1,3,5-triazine (MEA-Triazine) contains high nitrogen content. Also on reaction with hydrogen sulphide gas, they produce methylamine and monoethanolamine as side products, respectively. Methylamine is a gaseous, flammable, highly corrosive with low flash point (−30° C.) low molecular weight amine. Methylamine also causes top of line (TOL) corrosion. MEA-Triazine has identified with high risk of fouling and categorized as fatal by inhalation in European legislation. This is both an environmental and a process priority to replace this product with non-triazines and non-fouling compounds to overcome the drawbacks associated with the existing chemistries across the globe.

Besides, in some cases where a crude oil has been treated with triazines, the formed amines can stabilize emulsions and deteriorate desalter performance. They can also contribute to chloride salt formation in distillation towers, with subsequent increase in corrosion and fouling potential. Another drawback from triazine is its primary reaction product, dithiazine, which can undergo further reaction to form an amorphous dithazine and can also contribute to deposition and equipment fouling.

There is a need for a hydrogen sulphide and/or mercaptan scavenger without any nitrogen content and which can have satisfying scavenging properties.

### SUMMARY OF THE INVENTION

The present invention is directed to a composition comprising the reaction product of at least one phenol compound with aldehyde(s), wherein the phenol compound(s) reply to formula (II):

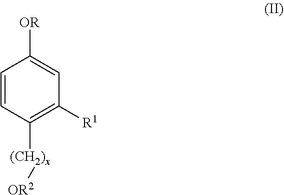

wherein
R represents a hydrogen atom or a hydrocarbyl group comprising from 1 to 30 carbon atoms,
x is 1 or 2,
R¹ is a linear alkyl or alkenyl group having 15 carbon atoms if x is 1,
R¹ is hydrogen if x is 2,
R² represents a hydrogen atom or a hydrocarbyl group comprising from 1 to 30 carbon atoms.

According to an embodiment, the phenol compound is selected from tyrosol, 3-pentadecylphenol and cardanol.

According to an embodiment, the aldehydes are selected from formaldehydes and paraformaldehydes.

According to an embodiment, the reaction product comprises at least one compound of formula (I):

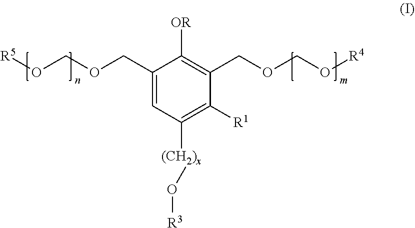

Wherein
R represents a hydrogen atom or a hydrocarbyl group comprising from 1 to 30 carbon atoms, n and m are, independently to each other, an integer ranging from 0 to 6,
x is 1 or 2,
R¹ is a linear alkyl or alkenyl group having 15 carbon atoms if x is 1,
R¹ is hydrogen if x is 2,
R³ is identical to R2 defined in any one or claim 1 to 3 or R³ represents —(CH₂O)_{y}H or a linear or branched alkyl group having from 10 to 40 carbon atoms,
y is an integer ranging from 1 to 5,
R⁴ and R⁵ represent, independently to each other, a hydrogen atom or a hydrocarbyl group comprising from 1 to 30 carbon atoms.

According to an embodiment, the compound of formula (I) is selected from compounds of formula (Ib):

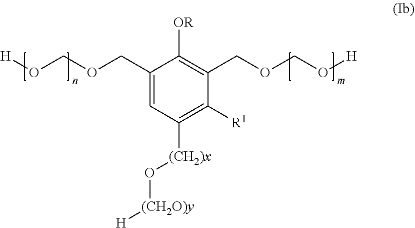

Wherein:
R is a hydrogen atom or a linear, branched, cyclic, alkyl or alkenyl group comprising from 1 to 16 carbon atoms,
R¹ is hydrogen,
x ranges from 2 to 4, preferably from 2 to 3, more preferably x is 2,
n and m are identical and ranges from 0 to 4, preferably from 0 to 2, more preferably from 0 to 1,
y ranges from 1 to 4, preferably from 1 to 3, more preferably from 0 to 2.

According to embodiment, in the formula (I) or (Ib), R¹ is hydrogen. Preferably, according to this embodiment:
x ranges from 2 to 4, preferably from 2 to 3, more preferably x is 2, and/or
n and m are identical and ranges from 0 to 4, preferably from 0 to 2, more preferably from 0 to 1, and/or
y ranges from 1 to 4, preferably from 1 to 3, more preferably from 0 to 2.

According to an embodiment of the invention, the composition comprises:
from 20 to 98% wt of the reaction product, and
from 2 to 80% wt of solvent(s),

based on the total weight of the composition.

The invention also relates to the use of the composition according to the invention, to scavenge hydrogen sulphide and mercaptans in streams selected from hydrocarbon streams and water-containing streams.

Preferably, the reaction product is added in an amount of at least 1% wt, based on the total weight of hydrogen sulphide and mercaptans in the stream.

Preferably, the streams are selected from hydrocarbon-containing streams such as liquefied petroleum gas (LPG), finished fuels, crude and heavy residual oils, natural gas asphalt, oil-based drilling fluids, and water-containing streams such as water-based drilling fluids.

The present invention is also directed to a hydrocarbon-containing composition comprising hydrocarbons and the scavenging composition according to the invention.

Finally, the invention is directed to a water-containing stream comprising water and the scavenging composition according to the invention.

Unlike triazines, the scavenging compositions according to the invention will not have a tendency to produce solid reaction by-products, based on the chemical nature of reactants, product's and reaction products. In addition, this proposed solution will have a minimal impact on pH, which reduces associated mineral scaling. This combination of benefits enhances production and minimizes downtime linked to pipeline cleaning and solids' removal.

The compounds of the invention further show an improved storage stability.

Additionally, the compounds of the invention are obtained from compounds having a reduced or zero toxicity.

### DETAILED DESCRIPTION OF THE INVENTION

The present invention is directed to the reaction product of a phenol compound with aldehyde(s), and to composition comprising said reaction product.

More particularly, the present invention is directed to the reaction product of one or more phenol compounds of formula (II):

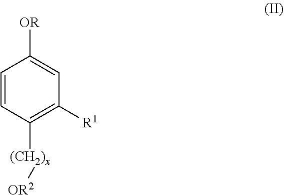

wherein
R represents a hydrogen atom or a hydrocarbyl group comprising from 1 to 30 carbon atoms,
x is 1 or 2,
R¹ is a linear alkyl or alkenyl group having 15 carbon atoms if x is 1,
R¹ is hydrogen if x is 2,
R² represents a hydrogen atom or a hydrocarbyl group comprising from 1 to 30 carbon atoms, with aldehyde(s).

Within the meaning of the present invention, the expression “reaction product of a phenol compound with aldehydes” does not encompass phenol-aldehyde resins containing oligomers of phenol compounds. In other words, the reaction product of the invention is different from a phenol-aldehyde resin containing at least two phenol units.

Within the meaning of the present invention, a “hydrocarbyl group” is a linear, branched or cyclic group that can be aliphatic or aromatic and when it is a cyclic group, the cycle(s) can be substituted by linear or branched groups. A hydrocarbyl group comprises carbon atoms and hydrogen atoms and optionally heteroatoms selected from oxygen, sulfur, halogen, silicon, nitrogen, preferably selected from oxygen, sulfur, halogen, silicon. According to a preferred embodiment, the hydrocarbyl group within the meaning of the present invention consists in carbon atoms and hydrogen atoms.

According to a preferred embodiment, the hydrocarbyl group within the meaning of the present invention is an aliphatic group that preferably consists in carbon atoms and hydrogen atoms.

According to a preferred embodiment, the hydrocarbyl group within the meaning of the present invention is an alkyl or an alkenyl group.

Within the meaning of the present invention, an alkyl group is a saturated group consisting in carbon atoms and hydrogen atoms, that can be linear or branched.

Within the meaning of the present invention, an alkenyl group is an unsaturated group consisting in carbon atoms and hydrogen atoms, that can be linear or branched. The alkenyl group can be mono-unsaturated or poly-unsaturated.

According to an embodiment, R is selected from hydrogen, alkyl group having from 1 to 30 carbon atoms, alkenyl group having from 2 to 30 carbon atoms, cycloalkyl group having from 5 to 30 carbon atoms optionally substituted by an alkyl group having from 1 to 12 carbon atoms, aryl group having from 6 to 30 carbon atoms optionally substituted by an alkyl group having from 1 to 12 carbon atoms, heterocyclic group having from 1 to 30 carbon atoms optionally substituted by an alkyl group having from 1 to 12 carbon atoms, propylene glycol, ethylene glycol.

According to an embodiment, the phenol compound replies to formula (II) wherein:
R represents a hydrogen atom,
x is 1 or 2,
R¹ is a linear alkyl or alkenyl group having 15 carbon atoms if x is 1,
R¹ is hydrogen if x is 2,
R² represents a hydrogen atom or a linear or branched alkyl group comprising from 4 to 20 carbon atoms.

According to an embodiment, the phenol compound is selected from tyrosol, tyrosol-derived compounds, cardanol, and 3-pentadecylphenol and mixtures thereof.

Within the meaning of the present invention, a tyrosol-derived compound can be a compound obtained by alkoxylation of one or two of the hydroxyl functions of the tyrosol. Among tyrosol-derived compounds, mention may be made of 4-(2-(alkoxy)ethyl)phenol, wherein the alkyl group of the alkoxy function can be selected from linear or branched alkyl or alkenyl groups comprising from 4 to 20 carbon atoms.

As an example, alkoxylation of tyrosol can be illustrated by the following scheme:

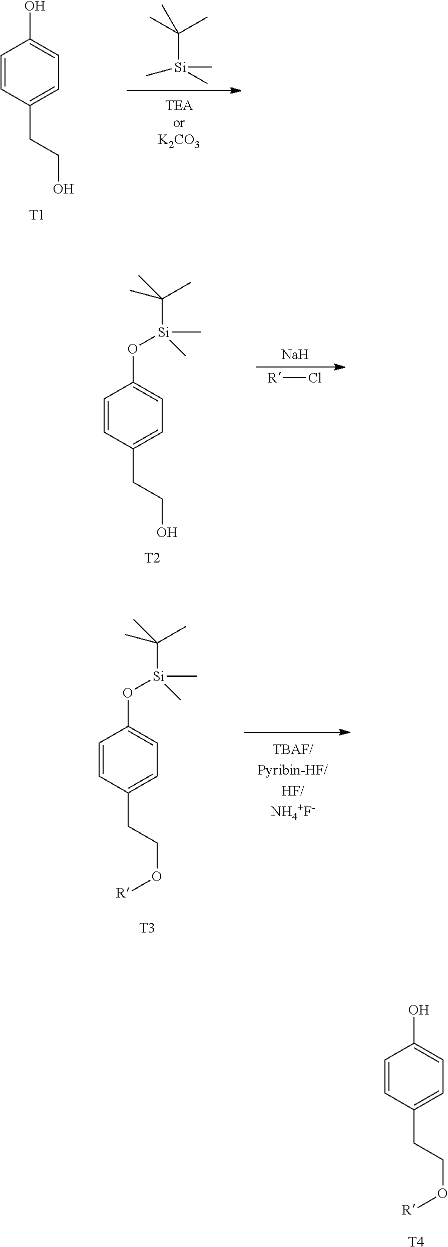

Wherein R′ can be a linear or branched alkyl group having from 4 to 20 carbon atoms.
In a typical synthetic route, tyrosol (T1) was reacted with tert-butyldimethylsilyl chloride (TBDM-Cl) in the presence of mild bases like, triethylamine (TEA) or potassium carbonate (K2CO3) under nitrogen atmosphere. Selective protection of the phenolic —OH was obtained by getting the mono-silyl ether (T2) of Tyrosol. In the second step, which was followed as Williamson's synthesis, under basic conditions, using sodium hydride (NaH) and chloride derivative of the respective alkyl chain, reaction of (T2) with alkyl-halide (typically alkyl chloride) afforded the O-alkylated mono-silyl ether (T3) of Tyrosol. In the final step, deprotection of the mono-silyl ether was achieved by employing either TBAF/Pyridin-HF/HF/NH⁴F⁻, to afford the final desired product (T4) in good yields.

According to an embodiment, the phenol compound of formula (II) is selected from tyrosol, 4-(2-(2-ethylhexyloxy)ethyl)phenol, 4-(2-dodecyloxy)ethyl)phenol, cardanol and 3-pentadecylphenol and mixture thereof.

The phenol compound can be commercially available. The phenol compound can also be prepared starting from tyrosol, for example by alkoxylation of tyrosol.

The cardanol may be originated from Cashew Nut Shell Liquid (CNSL). Technical CNSL are commercially available and generally comprises about 60-65% wt of cardanol, 10-15% wt of cardol, 0-5% wt of 2-méthyl cardol, and 10-25% wt of other materials (like oligomers and polymers), based on the total weight of the technical CNSL. According to an embodiment of the invention, the reaction product is obtained by contacting technical CNSL with aldehydes.

According to an embodiment, the phenol compound used to prepare the reaction products of the invention is of natural origin.

According to an embodiment, the reaction product is obtained from a mixture of phenol compounds replying to formula (II).

In particular, according to an embodiment wherein R¹ is a linear alkyl or alkenyl group having 15 carbon atoms, the mixture of phenol compounds can comprise:
At least one phenol compound replying to formula (II) wherein R¹ an alkyl group and
At least one phenol compound replying to formula (II) wherein R¹ is an alkenyl group.

According to an embodiment wherein R¹ is a linear alkyl or alkenyl group having 15 carbon atoms, the mixture of phenol compounds can comprise:
At least one phenol compound replying to formula (II) wherein R¹ is an alkyl group and
At least one phenol compound replying to formula (II) wherein R¹ is an alkenyl group having a unique unsaturation, and
At least one phenol compound replying to formula (II) wherein R¹ is an alkenyl group having two unsaturations.

According to an embodiment wherein R¹ is a linear alkyl or alkenyl group having 15 carbon atoms, the mixture of phenol compounds can comprise:
At least one phenol compound replying to formula (II) wherein R¹ is an alkyl group and
At least one phenol compound replying to formula (II) wherein R¹ is an alkenyl group having a unique unsaturation, and
At least one phenol compound replying to formula (II) wherein R¹ is an alkenyl group having two unsaturations, and
At least one phenol compound replying to formula (II) wherein R¹ is an alkenyl group having three unsaturations.

According to an embodiment, the aldehyde(s) are selected from formaldehyde(s) paraformaldehyde(s), and mixture thereof.

According to an embodiment of the invention, the reaction product comprises at least one compound of formula (I):

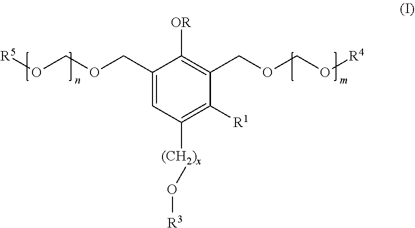

### Wherein

R represents a hydrogen atom or a hydrocarbyl group comprising from 1 to 30 carbon atoms,
n and m are, independently to each other, an integer ranging from 0 to 6,
x is 1 or 2,
R¹ is a linear alkyl or alkenyl group having 15 carbon atoms if x is 1,
R¹ is hydrogen if x is 2,
R³ is identical to R² defined above for the compound of formula (II) or R³ represents -(CH₂O)_{y}H or a linear or branched alkyl group having from 10 to 40 carbon atoms,
y is an integer ranging from 1 to 5,
R⁴ and R⁵ represent, independently to each other, a hydrogen atom or a hydrocarbyl group comprising from 1 to 30 carbon atoms.

According to an embodiment, in formula (I):
R is a hydrogen atom or a linear, branched, cyclic, alkyl or alkenyl group comprising from 1 to 16 carbon atoms, and/or
R³ is -(CH₂O)_{y}H or a linear or branched alkyl having from 4 to 20 carbon atoms, preferably from 6 to 16 carbon atoms, and/or
R⁴ and R⁵ are identical, preferably are a hydrogen atom, and/or
n and m are identical and ranges from 0 to 4, preferably from 0 to 2, more preferably from 0 to 1, and/or
y ranges from 0 to 4, preferably from 0 to 3, more preferably from 0 to 2.

According to an embodiment, in formula (I):
R is a hydrogen atom, and/or
R³ is —(CH₂O)_{y}H, and/or
R⁴ and R⁵ are a hydrogen atom, and/or
n and m are identical and ranges from 0 to 2, and/or
y ranges from 0 to 2.

Typically, if the phenol compound is cardanol, R¹ is the hydrocarbon chain of the aryl ring of the cardanol: —(CH₂)₇—CH═CH—CH₂—CH═CH—CH₂—CH═CH₂, or a partially or totally hydrogenated group. According to an embodiment, R¹ represents —(CH₂)₇—CH═CH—CH₂—CH═CH—CH₂—CH═CH₂ or a linear alkyl group having 15 carbon atoms. It can be noted that the reaction can also be obtained from a mixture of phenol compounds derived from cardanol, i.e. a mixture of compounds of formula (II) differing by the R¹ group. According to an embodiment, the mixture of phenol compounds comprises:
from 1 to 40% wt of phenol compound(s) replying to formula (II) wherein R is H and R¹ is an alkyl group having 15 carbon atoms, and
from 1 to 80% wt of phenol compound(s) replying to formula (II) wherein R is H and R¹ is —(CH₂)₇—CH═CH—(CH₂)₅—CH₃, and
from 1 to 40% wt of phenol compound(s) replying to formula (II) wherein R is H and R¹ is —(CH₂)₇—CH═CH—CH₂—CH═CH—(CH₂)₂—CH₃, and
from 5 to 95% wt of phenol compound(s) replying to formula (II) wherein R is H and R¹ is —(CH₂)₇—CH═CH—CH₂—CH═CH—CH₂—CH═CH₂.

According to an embodiment of the invention, the reaction product is obtained from tyrosol and the reaction product comprises at least one compound of formula (I), wherein:
R is a hydrogen atom or a linear, branched, cyclic, alkyl or alkenyl group comprising from 1 to 16 carbon atoms, and/or
R² is —(CH₂O)_{y}H, and/or
R¹ is hydrogen, and/or
x ranges from 2 to 4, preferably from 2 to 3, more preferably x is 2, and/or
n and m are identical and ranges from 0 to 4, preferably from 0 to 2, more preferably from 0 to 1, and/or
y ranges from 0 to 4, preferably from 0 to 3, more preferably from 0 to 2.

According to an embodiment, the compound of formula (I) is selected from compounds of formula (Ia):

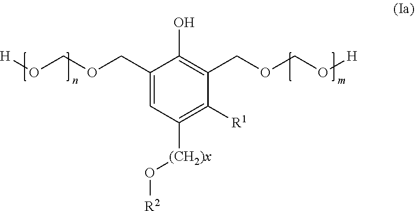

Wherein:
R² is —(CH₂O)_{y}H, and/or
R¹ is hydrogen, and/or
x ranges from 2 to 4, preferably from 2 to 3, more preferably x is 2, and/or
n and m are identical and ranges from 0 to 4, preferably from 0 to 2, more preferably from 0 to 1, and/or
y ranges from 1 to 4, preferably from 1 to 3, more preferably from 0 to 2.

According to an embodiment, the compound of formula (I) is selected from compounds of formula (Ib):

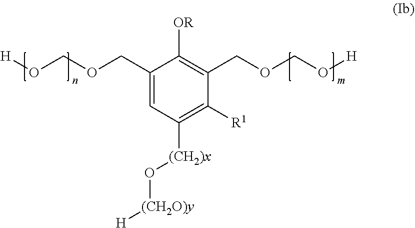

Wherein:
R is a hydrogen atom or a linear, branched, cyclic, alkyl or alkenyl group comprising from 1 to 16 carbon atoms
R¹ is hydrogen,
x ranges from 2 to 4, preferably from 2 to 3, more preferably x is 2,
n and m are identical and ranges from 0 to 4, preferably from 0 to 2, more preferably from 0 to 1,
y ranges from 1 to 4, preferably from 1 to 3, more preferably from 0 to 2.

According to an embodiment, the compound of formula (I) is selected from compounds of formula (Ic):

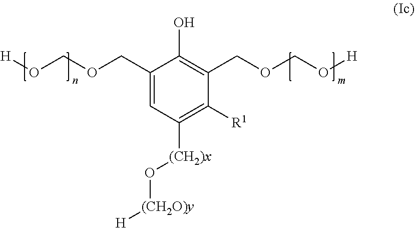

Wherein:
R¹ is hydrogen,
x ranges from 2 to 4, preferably from 2 to 3, more preferably x is 2,
n and m are identical and ranges from 0 to 4, preferably from 0 to 2, more preferably from 0 to 1,
y ranges from 1 to 4, preferably from 1 to 3, more preferably from 0 to 2.

Another object of the present invention is a process for manufacturing the reaction product of the invention.

The process for manufacturing the reaction product of the invention can comprises a step of contacting aldehyde(s) with a phenol compound of formula (II), in the presence of a base, preferably selected from potassium hydroxide, sodium hydroxide and metal alkoxide, preferably potassium hydroxide.

The process for manufacturing the reaction product of the invention can be performed with a molar ratio phenol/aldehyde ranging from 1/20 to 1/1, preferably from 1/10 to 1/1, more preferably from 1/8 to 1/2. The higher the amount of formaldehyde is, the higher the values of n, m and y will be, in the formulas (I), (Ia), (Ib) and (Ic).

The process for manufacturing the reaction product of the invention can be performed in one or several steps, by successive addition of aldehyde(s).

The phenol compound of formula (II) that reacts with the aldehyde(s) may be submitted to a step of alkoxylation before the contact with aldehyde(s).

The phenol compound of formula (II) that reacts with the aldehyde(s) may be submitted to a step of hydrogenation, before the contact with aldehyde(s) in order to partially or fully hydrogenate the carbon-carbon double bond(s) of the hydrocarbyl group of the compound of formula (II).

Examples of synthesis of the reaction product of the invention are given in the experimental part.

The reaction product of the invention can be used to scavenge hydrogen sulphide and/or mercaptans in hydrocarbons. The reaction product is also named “H₂S scavenger” in the present invention.

The reaction product can be added into hydrocarbons via a composition, named a scavenging composition, comprising said reaction product. According to an embodiment, the composition according to the invention comprises at least 20% wt, preferably at least 30% wt, more preferably at least 40% wt, of the reaction product as defined in the present invention, based on the total weight of the composition.

According to an embodiment, the reaction product is added in an amount of at least 1% wt, preferably of at least 5% wt, more preferably of at least 10% wt, based on the total weight of hydrogen sulphide and mercaptans in the hydrocarbon stream. According to an embodiment, the reaction product is added in an amount of from 1 to 70% wt, preferably from 5 to 60% wt, more preferably from 10 to 50% wt, based on the total weight of hydrogen sulphide and mercaptans in the hydrocarbon stream.

According to an embodiment, the composition according to the invention comprising the reaction product defined above, further comprises one or more solvents.

According to an embodiment, the composition comprises:
from 20 to 98% wt, preferably from 25 to 90% wt, more preferably from 30 to 80% wt, of the reaction product defined in the present invention, and
from 2 to 80% wt, preferably from 10 to 75% wt, more preferably from 20 to 70% wt, of solvent(s), based on the total weight of the composition.

The solvent can be selected in order to have a solution or a dispersion of the reaction product of the invention. The solvent can thus be either oil soluble, or water soluble or the solvent can have a dual solubility. Preferably, if the reaction product is obtained from tyrosol, the solvent if any will be preferably a water-soluble solvent, such as water. Preferably, if the reaction product is obtained from cardanol, the solvent if any will be preferably an oil soluble solvent, such as an aromatic solvent.

Preferably, the solvent is selected from poly alkyl ethers, aliphatic or aromatic solvents, such as N-methylpyrrolidone, butyl carbitol, xylene, toluene, and benzene. Typically, the solvent does not allow to scavenger or neutralize hydrogen sulphide or mercaptans in hydrocarbon streams. However, depending on the final use of the scavenging composition, a solvent having a dual solubility, i.e. a water solubility and a solubility in hydrocarbons, can be preferred. Butyl carbitol is a suitable solvent since it has this dual solubility.

Within the meaning of the present invention, a compound is said “soluble” in water or water-soluble if it can form a solution in water. A compound is said “not-soluble” in water if it can form a dispersion in water.

The scavenging compositions according to the invention can be used in both water soluble and oil-soluble solvents.

Depending on the media in which they are intended to be used, the reaction product of the invention can be dissolved in adapted solvents. The media can be hydrocarbons or water-containing compositions such as water-based muds.

Hydrocarbons can be selected from liquefied petroleum gas (LPG), finished fuels such as diesel, kerosene or gasoline, crude and heavy residual oils, and asphalt.

Once the H₂S scavenger(s) is/are added into hydrocarbons, a hydrocarbon-containing composition is obtained. The hydrocarbon-containing composition can be either a single-phase hydrocarbon composition or a multiphase system comprising oil/water or oil/water/gas or gas/water.

In multiphase systems comprising water, it is preferred to use a water-soluble scavenger dissolved in a water-soluble solvent. Examples of water-soluble solvents include water, glycols, butylcarbitol. Water-soluble scavengers are among the most common scavengers and are often the product of choice for applications at temperatures below 200° F. (93° C.). Economical costs and fast reaction rates make them attractive options. Water soluble scavengers are preferred for use in LPG, residues and crude oils and for use in water-based muds.

Preferably, the scavenging composition comprises:
from 20 to 98% wt of compound(s) of formula (I), preferably from 30 to 70% wt of compound(s) of formula (I), more preferably from 40 to 60% wt of compound(s) of formula (I), and
from 2 to 80% wt of solvent(s), preferably from 30 to 70% wt of solvent(s), more preferably from 40 to 60% wt of solvent(s),

based on the total weight of the scavenging composition.

According to an embodiment, the scavenging composition comprises:
from 20 to 98% wt of compound(s) of formula (I), preferably from 30 to 70% wt of compound(s) of formula (I), more preferably from 40 to 60% wt of compound(s) of formula (I), and
from 2 to 80% wt of water, preferably from 30 to 70% wt of water more preferably from 40 to 60% wt of water,

based on the total weight of the scavenging composition.

According to an embodiment, the reaction product comprises at least one compound selected from the compounds of formula (IIA), (IIB), (IIIA) or (IIIB):

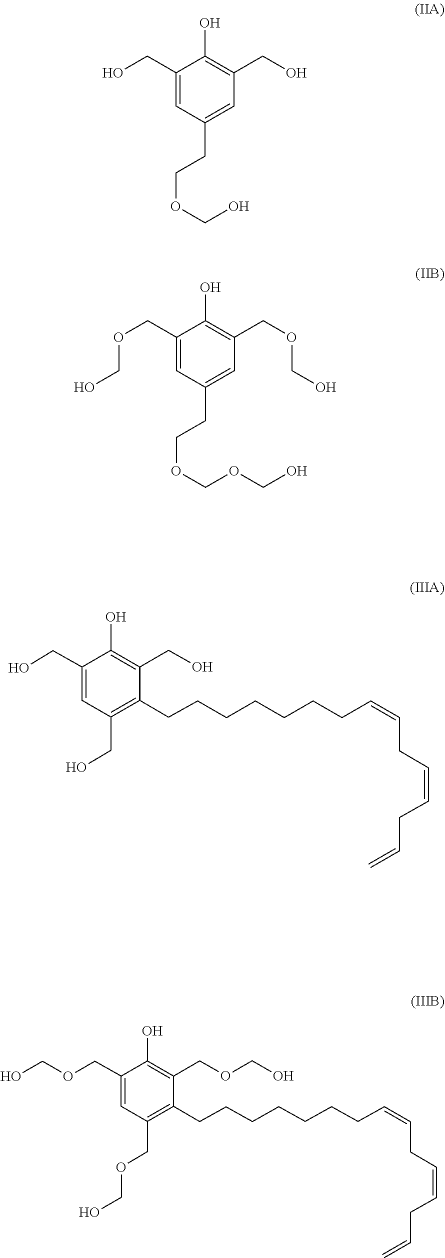 [SMILES: OCOCCc1cc(CO)c(O)c(CO)c1 ; OCOCOCCc1cc(COCO)c(O)c(COCO)c1 ; C=CC/C=C\C/C=C\CCCCCCCc1c(CO)cc(CO)c(O)c1CO ; C=CC/C=C\C/C=C\CCCCCCCc1c(COCO)cc(COCO)c(O)c1COCO]

being noted that the C15 chain of the compounds of formula (IIIA) and (IIIB) can be partially or totally hydrogenated.

Typically, compounds of formulas (IIA) and (IIB) are water-soluble and maybe used in a scavenging composition comprising one or more water-soluble solvents, such as water. 10 Typically, compounds of formulas (IIIA) and (IIIB) are oil-soluble and may be used in a scavenging composition comprising one or more oil-soluble solvents, such as aromatic solvents, or solvents having a dual solubility, such as butyl carbitol.

### Hydrocarbon-Containing Composition

The invention is also directed to a hydrocarbon-containing composition comprising:
at least 70% wt of hydrocarbon(s), and
at least 10 ppm by weight of reaction product(s) as defined in the invention,
optionally at least 10 ppm by weight of one or more solvent(s) different from the hydrocarbon(s).

Preferably, the reaction product comprises one or more of the features defined above in relation to the composition of the invention.

Preferably, the solvent comprises one or more of the features defined above in relation to the composition of the invention.

The hydrocarbon-containing composition can be prepared by adding the scavenging composition of the invention comprising the reaction product alone or in the presence of solvent(s) into hydrocarbons.
According to an embodiment of the present invention, the weight ratio H₂S:scavenging composition ranges from 1:2 to 1:0.05, preferably from 1:1.5 to 1:0.1, more preferably from 1:1 to 1:0.3, even more preferably from 1:0.8 to 1:0.4 and advantageously from 1:0.8 to 1:0.4. In this ratio, H₂S represents the amount of hydrogen sulphide in the hydrocarbons, before contacting with the scavenging composition of the invention.

Hydrocarbons can be selected from natural gas, liquefied petroleum gas (LPG), finished fuels such as diesel, kerosene or gasoline, crude and heavy residual oils, and asphalt.

Hydrocarbons contain H₂S and/or mercaptans, in an amount for example ranging from 1 to 10 000 ppm. Mercaptans that can be removed from hydrocarbon streams within the framework of the present invention may be C₁-C₆ mercaptans, such as C₁-C₄ mercaptans. The scavenging composition of the invention may represent from 0.0005 to 5% by weight of the total weight of the hydrocarbon-containing composition.

The scavenging compositions according to the invention can be used in LPG when contaminated with H₂S, e.g. after a unit upset. Presence of H₂S in LPG may cause metal corrosion and a potential health hazard to consumers. Water-soluble scavengers are generally recommended for LPG because they will separate completely from the hydrocarbon and prevent contamination of the LPG with materials of lower volatility. The presence of lower-volatility components in LPG is undesirable because these materials do not burn as well and could cause injector plugging and fouling on burner tips.

The scavenging compositions according to the invention can be used in finished fuels such as gasoline, kerosene and diesel which are required to be noncorrosive. Oil-soluble, nonreversible H₂S scavengers are typically the product of choice because they will not adversely affect critical fuel properties and will not add water (and possibly a haze) to a finished fuel.

The scavenging compositions according to the invention can be used in crude and heavy residual oils contain significant concentrations of H₂S as a natural component and/or as a result of thermal cracking processes that break apart high molecular weight sulfur-containing compounds to generate H₂S.

The scavenging compositions according to the invention can be used in asphalt, which contains extremely high levels of H₂S, often exceeding 1% (10,000 ppm). Asphalt is the heaviest of the products coming out of the refinery and typically the product in which sulfur compounds concentrate. Because of the high viscosity of asphalt, it must be stored at high temperatures (300 to 400° F.). These temperatures promote cracking of sulfur-containing compounds and formation of H₂S. Moreover, asphalt has a high vapor:liquid partition coefficient (400:1), meaning that H₂S tends to collect in the vapor phase. The combination of high temperatures, high H₂S concentrations, and high viscosity makes asphalt challenging to treat. It is especially critical to lower the H₂S content because asphalt is shipped by rail car and tank truck, and exposure of personnel and consumers is a real concern. Because of the elevated temperatures of asphalt applications, water-soluble scavengers generally are not suitable; rather, oil-soluble carriers for scavengers are preferred.

The scavenging compositions according to the invention may also be used in drilling fluids, in oil-based drilling fluids or in water-based drilling fluids, for drilling applications.

The present invention is also directed to a drilling fluid comprising the scavenging composition of the invention, preferably in an amount of at least 10 ppm by weight, based on the total weight of the scavenging composition.

### Water-Containing Composition

The invention is also directed to a water-containing composition comprising:
at least 70% wt of water, and
at least 10 ppm by weight of the reaction product(s) as defined in the invention,
optionally at least 10 ppm by weight of one or more solvent(s) different from water.

Preferably, the reaction product comprises one or more of the features defined above in relation to the composition of the invention.

Preferably, the solvent comprises one or more of the features defined above in relation to the composition of the invention.

The water-containing composition can be prepared by adding the scavenging composition of the invention comprising the reaction product alone or in the presence of solvent(s) into a water-containing media. The water-containing media is preferably a water-based mud.

According to an embodiment, the water-containing composition of the invention is a water-based mud comprising the scavenging composition of the invention.

According to an embodiment of the present invention, the weight ratio H₂S:scavenging composition ranges from 1:2 to 1:0.05, preferably from 1:1.5 to 1:0.1, more preferably from 1:1 to 1:0.3, even more preferably from 1:0.8 to 1:0.4 and advantageously from 1:0.8 to 1:0.4. In this ratio, H₂S represents the amount of hydrogen sulphide in the hydrocarbons, before contacting with the scavenging composition of the invention.

The invention is thus particularly useful in order to reduce hydrogen sulphide amount of water-based drilling fluids.

### EXAMPLES

The invention is now described with the help of the following examples, which are not intended to limit the scope of the present invention, but are incorporated to illustrate advantages of the present invention and best mode to perform it. The following examples also demonstrate the synthesis of different reaction product of the invention and the effectiveness of H₂S scavengers of the invention. The reaction products of the invention can be characterized by IR, ¹H NMR and ¹³C NMR spectroscopic techniques.

### Example 1: Synthesis of Reaction Products of the Invention From Tyrosol

### Example 1a: Synthesis of (2-hydroxy-5-(2-(hydroxymethoxy)ethyl)-1,3-phenylene)dimethanol (IIA):

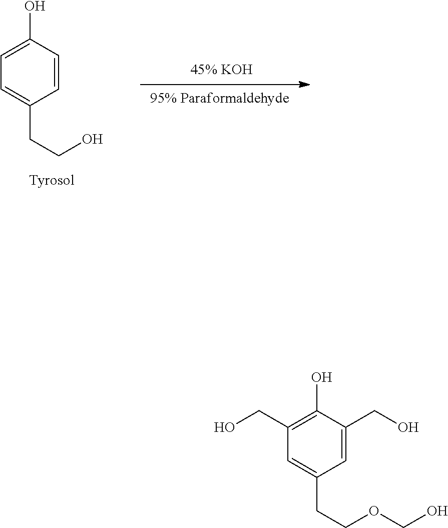 [SMILES: OCCc1ccc(O)cc1 ; OCOCCc1cc(CO)c(O)c(CO)c1]

It has been prepared according to the following process:
In a 250 mL four necked Round bottom flask 54 g (45%) aqueous potassium hydroxide solution was added to 60 g (0.43 moles) Tyrosol over a period of 30 minutes (exotherm up to 40° C. was observed) to get clear solution. This exotherm was controlled below 40° C., using bath chilling systems.
To this clear solution, 41.2 g (95%) Paraformaldehyde (1.3 mol) was added over 15 minutes. (No exotherm observed). We can also use 37% wt Aqueous formaldehyde. Reaction mass was transformed into slightly viscous slurry.
The temperature of reaction mass was raised to 60° C. and it was maintained further for 6 hours at 60° C. (Reaction mass became clear at 55° C.).
The excess of water from reaction mass was removed under reduced pressure at 60-65° C. to get 101 g of clear viscous liquid product.

### Example 1b: Synthesis of (((2-hydroxy-5-(2-((hydroxymethoxy)methoxy)ethyl)-1,3-phenylene)bis(methylene))bis(oxy))dimethanol (IIB):

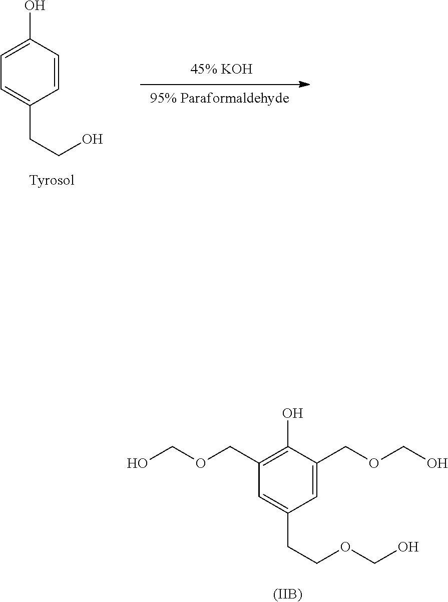 [SMILES: OCCc1ccc(O)cc1 ; OCOCCc1cc(COCO)c(O)c(COCO)c1 ; "(IIB)"]

It has been prepared according to the following process:
In a 250 mL four-necked R B flask 54 g (45%) aqueous potassium hydroxide, solution was added to 60 g (0.43 moles) of Tyrosol over 30 minutes. (Exotherm up to 40° C. was observed) to get clear solution. This exotherm was controlled below 40° C., using bath chilling systems.
To this clear solution, 82.4 g (95%) Paraformaldehyde (2.58 mol) (was added over 15 minutes. (No exotherm observed). Reaction mass transformed into slightly viscous stir-able slurry.
The temperature of reaction mass was raised to 60° C. and it was maintained further for 6 hours at 60° C. (Reaction mass became clear at 55° C.).
The excess of water from reaction mass was removed under reduced pressure at 60-65° C. to get 130 g of clear viscous liquid product.

### Example 2: Synthesis of Other Reaction Products of the Invention From 3-Pentadeca-8,11,14-trien-1-yl-phenol

### Example 2a: Synthesis of (2-hydroxy-4-((8E,11E)-pentadeca-8,11,14-trien-1-yl)benzene-1,3,5-triyl)trimethanol (IIIA):

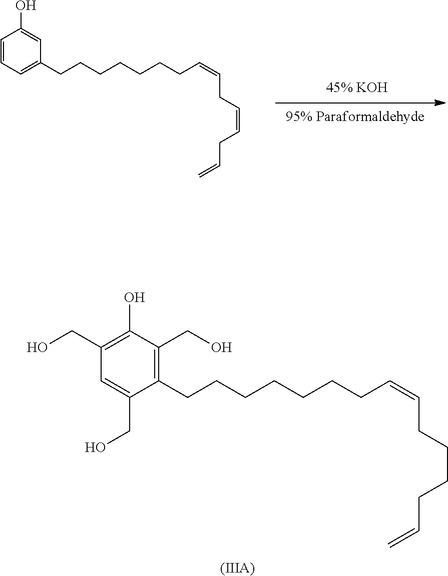 [SMILES: C=CC/C=C\C/C=C\CCCCCCCc1cccc(O)c1 ; C=CCCCC/C=C\CCCCCCCc1c(CO)cc(CO)c(O)c1CO ; "(IIIA)"]

It has been prepared according to the following process:
In a 250 mL four necked Round bottom flask 54 g (45%) methanolic potassium hydroxide solution was added to 128 g (0.43 moles) of 3-Pentadeca-8,11,14-trien-1-yl-phenol over a period of 30-60 minutes (exotherm up to 40° C. was observed) to get clear solution. This exotherm was controlled below 40° C., using bath chilling systems.
To this clear solution, 41.2 g (95%) Paraformaldehyde was added over 15-30 minutes. (No exotherm observed). Reaction mass was transformed into slightly viscous turbid slurry.
The temperature of reaction mass was raised to 60° C. and it was maintained further for 6 hours at 60° C. (Reaction mass became clear at 55° C.).
The excess of water and methanol from reaction mass was removed under reduced pressure at 60-65° C. to get 160 g of low melting solid product.

### Example 2b: Synthesis of 2-hydroxy-4-((8E,11E)-pentadeca-8,11,14-trien-1-yl)-1,3,5-phenylene)tris(methylene))tris(oxy))trimethanol (IIIB):

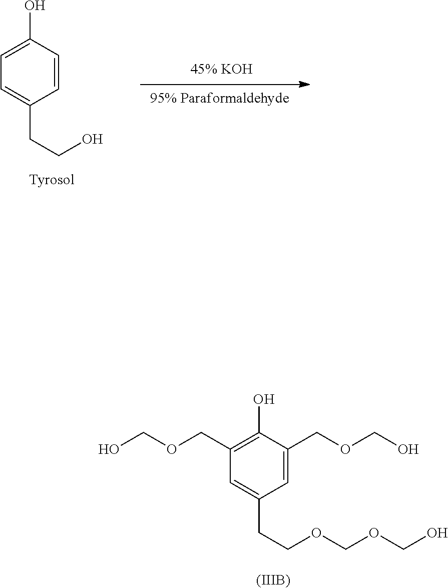

It has been prepared according to the following process:
In a 250 mL four necked Round bottom flask 54 g (45%) methanolic potassium hydroxide solution was added to 128 g (0.43 moles) of 3-Pentadeca-8,11,14-trien-1-yl-phenol over a period of 30-60 minutes (exotherm up to 40° C. was observed) to get clear solution. This exotherm was controlled below 40° C., using bath chilling systems.
To this clear solution, 82.4 g (95%) Paraformaldehyde was added over 15-30 minutes. (No exotherm observed). Reaction mass transformed into slightly viscous stir-able slurry.
The temperature of reaction mass was raised to 60° C. and it was maintained further for 6 hours at 60° C. (Reaction mass became clear at 55° C.).
The excess of water and methanol from reaction mass was removed under reduced pressure at 60-65° C. to get 200 g of low melting solid product.

### Example 3: Synthesis of Reaction Products From 3-pentadecyl-phenol

### Example 3a: Synthesis of (2-pentadecyl-4-hydroxybenzene-1,3,5-triyl)trimethanol (IVA):

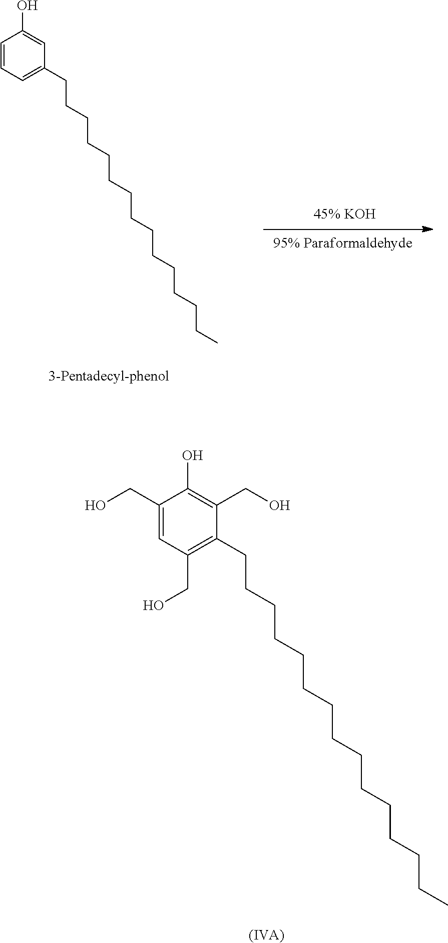 [SMILES: CCCCCCCCCCCCCCCc1cccc(O)c1 ; CCCCCCCCCCCCCCCc1c(CO)cc(CO)c(O)c1CO ; "(IVA)"]

It has been prepared according to the following process:
In a 250 mL four necked Round bottom flask 54 g (45%) methanolic potassium hydroxide solution was added to 130 g (0.43 moles) of 3-Pentadecyl-phenol over a period of 30 minutes (exotherm up to 40° C. was observed) to get clear solution. This exotherm was controlled below 40° C., using bath chilling systems. To this clear solution, 41.2 g (95%) Paraformaldehyde was added over 15 minutes. (No exotherm observed). Reaction mass was transformed into slightly viscous turbid slurry.
The temperature of reaction mass was raised to 60° C. and it was maintained further for 6 hours at 60° C. (Reaction mass became clear at 55° C.).
The excess of water and methanol from reaction mass was removed under reduced pressure at 60-65° C. to get 170 g of low melting solid product.

### Example 3b: Synthesis of (((2-hydroxy-4-pentadecylbenzene-1,3,5-triyl)tris(methylene))tris(oxy))trimethanol (IVB):

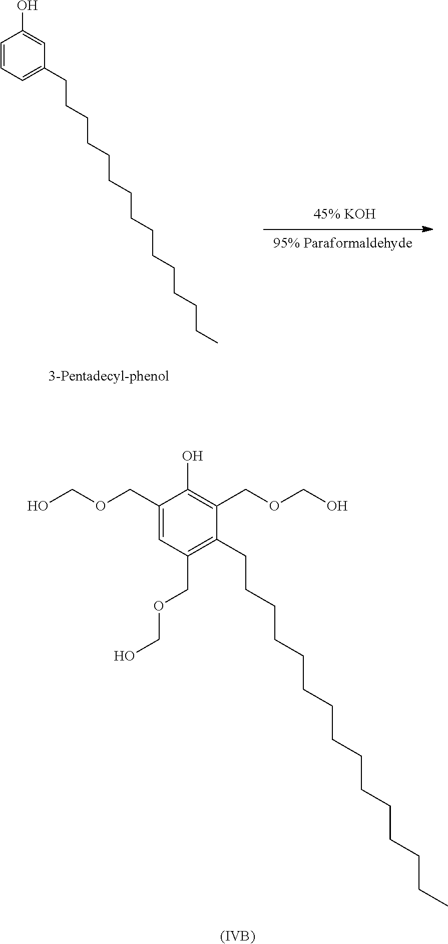 [SMILES: CCCCCCCCCCCCCCCc1cccc(O)c1 ; CCCCCCCCCCCCCCCc1c(COCO)cc(COCO)c(O)c1COCO ; "(IVB)"]

It has been prepared according to the following process:
In a 250 mL four-necked R B flask 54 g (45%) methanolic potassium hydroxide, solution was added to 130 g (0.43 moles) of 3-Pentadecyl-phenol over 30 minutes. (Exotherm up to 40° C. was observed) to get clear solution. This exotherm was controlled below 40° C., using bath chilling systems.
To this clear solution, 82.4 g (95%) Paraformaldehyde was added over 15-30 minutes. (No exotherm observed). Reaction mass transformed into slightly viscous stir-able slurry.
The temperature of reaction mass was raised to 60° C. and it was maintained further for 6 hours at 60° C. (Reaction mass became clear at 55° C.).
The excess of water and methanol from reaction mass was removed under reduced pressure at 60-65° C. to get 210 g of low melting solid product.

### Example 4: Synthesis of Reaction Products of the Invention From Alkoxylated Tyrosol

### Example 4a: Synthesis of 5-(2-(2-ethylhexyloxy)ethyl)-2-hydroxy-1,3-phenylene)dimethanol (VA):

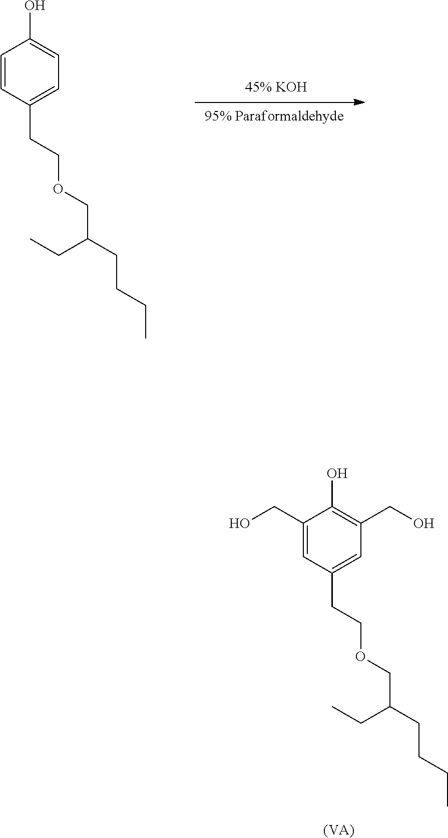 [SMILES: CCCCC(CC)COCCc1ccc(O)cc1 ; CCCCC(CC)COCCc1cc(CO)c(O)c(CO)c1 ; "(VA)"]

It has been obtained from the following process:
In a 250 mL four necked Round bottom flask 54 g (45%) methanolic potassium hydroxide solution was added to 107 g (0.43 moles) of 4-(2-(2-ethylhexyloxy)ethyl)-phenol (prepared by a similar process as the process described above for preparing compounds T4 wherein R′ is 2-ethylhexyl) over a period of 30 minutes (exotherm up to 40° C. was observed) to get clear solution. This exotherm was controlled below 40° C., using bath chilling systems.
To this clear solution, 41.2 g (95%) Paraformaldehyde was added over 15 minutes. (No exotherm observed). Reaction mass was transformed into slightly viscous turbid slurry.
The temperature of reaction mass was raised to 60° C. and it was maintained further for 6 hours at 60° C. (Reaction mass became clear at 55° C.).
The excess of water and methanol from reaction mass was removed under reduced pressure at 60-65° C. to get 130 g of low melting solid product.

### Example 4b: Synthesis of (((5-(2-(2-ethylhexyloxy)ethyl)-2-hydroxy-1,3-phenylene)bis(methylene))bis(oxy))dimethanol (VB):

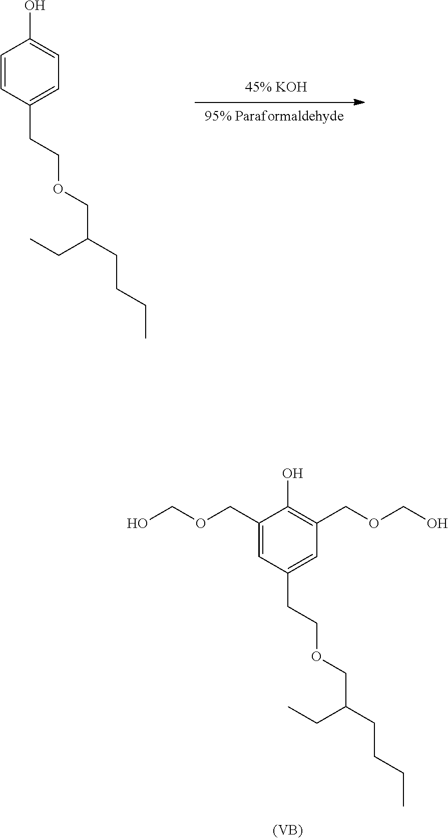 [SMILES: CCCCC(CC)COCCc1ccc(O)cc1 ; CCCCC(CC)COCCc1cc(COCO)c(O)c(COCO)c1 ; "(VB)"]

It has been prepared according to the following process:
In a 250 mL four necked Round bottom flask 54 g (45%) methanolic potassium hydroxide solution was added to 107 g (0.43 moles) of 4-(2-(2-ethylhexyloxy)ethyl)-phenol over a period of 30 minutes (exotherm up to 40° C. was observed) to get clear solution. This exotherm was controlled below 40° C., using bath chilling systems.
To this clear solution, 82.4 g (95%) Paraformaldehyde was added over 15-30 minutes. (No exotherm observed). Reaction mass transformed into slightly viscous stir-able slurry.
The temperature of reaction mass was raised to 60° C. and it was maintained further for 6 hours at 60° C. (Reaction mass became clear at 55° C.).
The excess of water and methanol from reaction mass was removed under reduced pressure at 60-65° C. to get 150 g of low melting solid product.

### Example 4c: Synthesis of (5-(2-(dodecyloxy)ethyl)-2-hydroxy-1,3-phenylene)dimethanol (VIA):

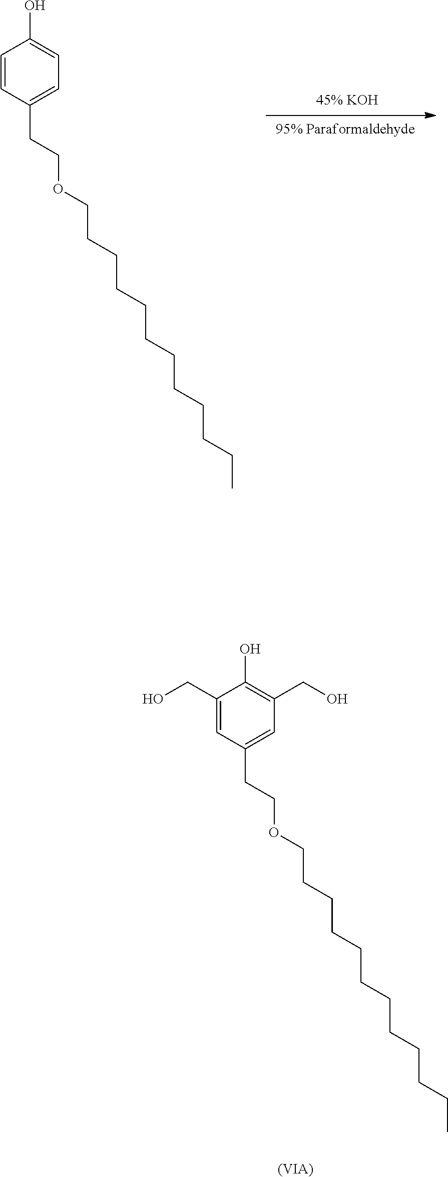 [SMILES: CCCCCCCCCCCCOCCc1ccc(O)cc1 ; CCCCCCCCCCCCOCCc1cc(CO)c(O)c(CO)c1 ; "(VIA)"]

It has been prepared according to the following process:
In a 250 mL four necked Round bottom flask 54 g (45%) methanolic potassium hydroxide solution was added to 107 g (0.43 moles) of 4-(2-(2-dodecyloxy)ethyl)-phenol (prepared by a similar process as the process described above for preparing compounds T4 wherein R′ is 2-ethylhexyl) over a period of 30 minutes (exotherm up to 40° C. was observed) to get clear solution. This exotherm was controlled below 40° C., using bath chilling systems.
To this clear solution, 41.2 g (95%) Paraformaldehyde was added over 15-30 minutes. (No exotherm observed). Reaction mass was transformed into slightly viscous turbid slurry.
The temperature of reaction mass was raised to 60° C. and it was maintained further for 6 hours at 60° C. (Reaction mass became clear at 55° C.).
The excess of water and methanol from reaction mass was removed under reduced pressure at 60-65° C. to get 150 g of low melting solid product.

### Example 4d: Synthesis of (((5-(2-(dodecyloxy)ethyl)-2-hydroxy-1,3-phenylene)bis(methylene))bis(oxy))dimethanol (VIB)

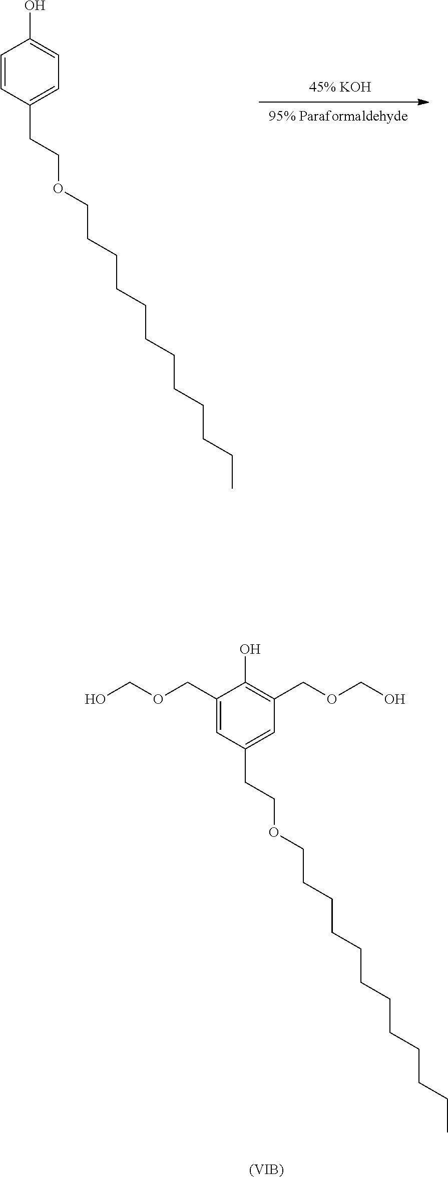 [SMILES: CCCCCCCCCCCCOCCc1ccc(O)cc1 ; CCCCCCCCCCCCOCCc1cc(COCO)c(O)c(COCO)c1 ; "(VIB)"]

It has been prepared according to the following process:
In a 250 mL four necked Round bottom flask 54 g (45%) methanolic potassium hydroxide solution was added to 131 g (0.43 moles) of 4-(2-(dodecyloxy)ethyl)-phenol over a period of 30 minutes (exotherm up to 40° C. was observed) to get clear solution. This exotherm was controlled below 40° C., using bath chilling systems.
To this clear solution, 82.4 g (95%) Paraformaldehyde was added over 15-30 minutes. (No exotherm observed). Reaction mass transformed into slightly viscous stir-able slurry.
The temperature of reaction mass was raised to 60° C. and it was maintained further for 6 hours at 60° C. (Reaction mass became clear at 55° C.).
The excess of water and methanol from reaction mass was removed under reduced pressure at 60-65° C. to get 180 g of low melting solid product.

### Example 5: Protocol of Experiment for the Measurement of H₂S Scavenging Ability of the Scavenging Compositions Under Modified ASTM D-5705 Conditions

ASTM D-5705 is recommended for measurement of Hydrogen sulfide in a vapor phase above the residual fuel oils. Performance evaluation of the various products and formulations developed as Hydrogen Sulfide Scavengers were evaluated using a modified ASTM D-5705 test method as detailed below:

In a typical experiment, 1 liter tin metal bottles with inner and outer caps were used to prepare and hold the test media. A media named “HC1” and having an initial boiling point higher than 120° C., a final boiling point higher than 250° C. (the difference between the final boiling point and the initial boiling point ranges from 40 to 50° C.) and a flash point above 100° C. with aromatic content less than 0.05% wt and a paraffin content of more than 75% wt has been used for the tests.

In a representative experimental set, a defined amount of H₂S saturated hydrocarbon solvent, typically between 2000 and 7000 ppm by weight of H₂S, was injected in 1 liter tin metal bottle pre-filled with 500 ml of dearomatized hydrocarbon solvent through the silicon septa fixed at the opening of the bottle using micro-syringe. The metal bottle was then kept on a reciprocating shaking machine for 5 min to allow proper mixing of the H₂S gas. The tin metal bottle was then kept in a water bath at 60° C. for two hours. After two hours, the tin metal bottle was taken out and cooled down to room temperature under running tap water and kept aside. An H₂S detecting tube (Dräger tube, with typical detection limit ranging from 100 to 70000 ppm by weight) was inserted in a rubber cork through a hole having the same diameter as the detecting tube. The sealed ends of the H₂S detecting tube were opened with an appropriate opener, one end of the tube being attached to Dräger pump. The silicon septa mounted at the opening of the tin metal bottles was removed and very quickly the rubber cork with H₂S detector tube was inserted inside the opening of the tin metal bottle. The H₂S gas in the vapor phase of the tin metal bottle was then pulled through the H₂S measuring tube using Dräger pump attached at the other end of the tube. The detector tube was removed after complete decompression of the pump. H₂S concentration was read from the tubes calibration scale (typically color change from colorless to brown). This reading was noted as a reference Blank reading of H₂S amount.

Further, same amount of H₂S containing dearomatized hydrocarbon solvent was injected into other tin metal bottles, which are pre-filled with 500 mL of the dearomatized hydrocarbon, and H₂S scavengers at different ratios of scavenger against H₂S, based on the Blank reading. Typical H₂S:scavenger ratios employed were 1:1, 1:0.9, 1:0.8, 1:0.7, 1:0.6, 1:0.5, 1:0.4, 1:0.3, 1:0.2 and 1:0.1. All the metal bottles were kept in a water bath for two hours at 60° C. Similar protocol was employed to measure the H₂S in the vapor phase of all the bottles as used to make the Blank reading. The difference between the Blank H₂S concentration and H₂S concentration observed with different concentrations of the scavenging products and formulations are noted as % scavenging. A higher % Scavenging with lower concentration of the scavenging product is considered as better H₂S scavenger for the set of experiment.

The protocol of measurement was repeated three times with each scavenging composition and the indicated percentage was calculated based on the average of the measurements.

### Example 6: Measurement of H₂S Scavenging Ability of the Scavenging Compositions of the Invention Under Modified ASTM D-5705 Conditions, as Detailed in Example 5

H₂S scavengers according to the invention correspond to the reaction products prepared according to examples 1 to 4. More specifically, the scavenger of formula (IIA) had been prepared according to example 1a and the scavenger of formula (IIB) had been prepared according to example 1b.

Table 1 below summarizes the scavenging compositions that were tested.

The concentration of each H₂S scavenger reported in Table 1 corresponds to the actual amount of active ingredient in the scavenging composition. As such, composition I1 comprises 50 wt% of a compound of formula (IIA) and composition I2 comprises 50% wt of a compound of formula (IIB).

TABLE 1
scavenging compositions (in wt % based on the total weight of the
composition)

| H₂S scavenging | active ingredient | | solvent |
|---|---|---|---|
| composition | chemical structure | amount (wt %) | Water (wt %) |
| 11 | Formula (IIA) | 50 | 50 |
| 12 | Formula (IIB) | 50 | 50 |

Table 2 below shows the percentage of H₂S reduction based on the measured H₂S amount in vapour phase after treatment with the H₂S scavenging compositions of the invention (I1 and I2), as measured according to the method detailed in example 5.

TABLE 2
Scavenging efficiency (% of H₂S reduction) of the scavenging compositions

| | % Scavenging Efficiency v/s Scavenger Dosages [modified ASTM D5705] | | | | | | | | | |
|---|---|---|---|---|---|---|---|---|---|---|
| Ref | 1:1 | 1:0.9 | 1:0.8 | 1:0.7 | 1:0.6 | 1:0.5 | 1:0.4 | 1:0.3 | 1:0.2 | 1:0.1 |
| I1 | 100 | 100 | 100 | 100 | 100 | 99 | 96 | 84 | 60 | 48 |
| I2 | 100 | 100 | 100 | 100 | 100 | 100 | 99 | 98 | 96 | 70 |

The results in Table 2 clearly show that the scavenging compositions of the present invention are extremely efficient to scavenger hydrogen sulphide in the hydrocarbon-containing media.

If we refer for example to the sample wherein the weight ratio H₂S:scavenging composition is 1:0.4, we can observe that more than 96% of the H₂S has been scavenged with the scavenging compositions I1 and I2 according to the invention.

### Example 7: H₂S Scavenging Measurement

In this example, a comparative scavenger C1 have been synthesized and evaluated for its H2S scavenging performances.
The comparative scavenger C1 have been prepared according to the process described in para. [65] of the document US 2018/0216013 with a phenol:formaldehyde molar ratio of 1:3, the obtained active compound replies to formula (C1):

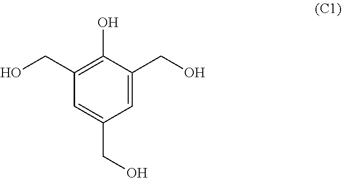 [SMILES: OCc1cc(CO)c(O)c(CO)c1]

The active compound is diluted in 50% wt of water.
The tested compositions of the invention are the compositions I1 and I2 as detailed in table 1.
Two hydrocarbon-containing media have been tested:
HC1 is a dearomatized hydrocarbon solvent having an initial boiling point higher than 120° C., a final boiling point higher than 250° C. (the difference between the final boiling point and the initial boiling point ranges from 40 to 50° C.) and a flash point above 100° C. with aromatic content less than 0.05% wt and a paraffin content of more than 75% wt, and
HC2 is a dearomatized hydrocarbon solvent having an initial boiling point higher than 120° C., a final boiling point lower than 250° C. (the difference between the final boiling point and the initial boiling point ranges from 20 to 35° C.) and a flash point above 65° C. with aromatic content less than 0.1% wt and a paraffin content of more than 75% wt.
   The H₂S measurement test is the same as detailed in example 5.
   The results are expressed in Tables 3 and 4 below

TABLE 3
H2S scavenging performances in the hydrocarbon-containing media HC1

| | H2S v/s Scavenger Dosage against % H2S Scavenged in HC1 | | | | | | | | | | |
|---|---|---|---|---|---|---|---|---|---|---|---|
| | 1:1 | 1:0.9 | 1:0.8 | 1:0.7 | 1:0.6 | 1:0.5 | 1:0.4 | 1:0.3 | 1:0.2 | 1:0.1 | 1:0.05 |
| C1 | 100 | 100 | 100 | 96 | 90 | 86 | 80 | 74 | 62 | 40 | 10 |
| I1 | 100 | 100 | 100 | 100 | 98 | 95 | 90 | 80 | 70 | 52 | 35 |
| I2 | 100 | 100 | 100 | 100 | 100 | 100 | 99 | 98 | 94 | 86 | 70 |

TABLE 4
H2S scavenging performances in the hydrocarbon-containing media HC2

| | H2S v/s Scavenger Dosage against % H2S Scavenged in HC2 | | | | | | | | | | |
|---|---|---|---|---|---|---|---|---|---|---|---|
| | 1:1 | 1:0.9 | 1:0.8 | 1:0.7 | 1:0.6 | 1:0.5 | 1:0.4 | 1:0.3 | 1:0.2 | 1:0.1 | 1:0.05 |
| C1 | 100 | 100 | 100 | 100 | 100 | 100 | 98 | 92 | 80 | 66 | 40 |
| I1 | 100 | 100 | 100 | 100 | 100 | 100 | 100 | 92 | 86 | 78 | 50 |
| I2 | 100 | 100 | 100 | 100 | 100 | 100 | 100 | 100 | 98 | 88 | 72 |

As demonstrated in the above tables 3 and 4, the scavenging products of the invention show a better efficiency than the scavenging product of the prior art.

### Example 8: Water-Based Mud Compositions

The H2S scavenger efficiency of the compounds of the invention has been evaluated in a water-based composition.

9.8 ppg of a water-based mud had been mixed with 130000 ppm of NaCl. The composition that has been obtained is detailed in table 5.

TABLE 5
composition of the water-based mud

| | Content (ppg) |
|---|---|
| Water | 334.25 |
| Sodium Chloride | 47 |
| Caustic Soda | 0.5 |
| Soda Ash | 0.5 |
| Viscosifier | 1.5 |
| Fluid loss | 6 |
| CO2 scavenger | 0.5 |
| Oxygen scavenger | 0.8 |
| Corrosion Inhibitor | 1.5 |
| Biocide | 0.5 |
| Calcium Carbonate | 18.26 |

The scavenging composition tested is the scavenging composition I1 as detailed in table 1. It has been added in 1% wt, based on the total weight of the water-based mud.
Hot-rolling of the mud is then performed in a multimixer during 16 hours at 150° F. for a water-based mud volume of 1 barrel at a speed of 11500 rpm.
Hot-rolled mud is then filtered to separate water and solids. The Test sample of the scavenging test is the filtered fluid.
The scavenging test has been performed in a Garret Gas Train instrument by OFITE instruments, according to the following protocol:
i. Ensure that the Garrett Gas Train is clean, dry and on a level surface.
ii. Add a mix of 1 ml of Sulphide source liquid (Sodium Hydrosulphide)+24 ml DI water in to chamber 1.
iii. Add 10 ml additional DI water
iv. Install top of gas train to lower chambers and tighten down all bolts
v. Attach flexible plastic tubing to gas train on exit nozzle
vi. Break off the ends of the dragger tube and attached to flexible tubing with the arrow on the dragger tube facing away from the gas train
vii. Insert a new carbon dioxide cartridge to the gas train pressure container. Screw down until a small amount of gas is released, then continue to tighten until gas stops escaping.
viii. Flow a small amount of gas through the system to purge it.
ix. Inject 10 ml of 0.5N Sulphuric Acid into chamber 1 using a hypodermic needle and syringe. Gently shake Garrett Gas train to mix liquid in chamber 1.
x. Inject 10 ml of the Test sample into chamber 1 using a hypodermic needle and syringe
xi. Restart gas flow to between 0.2 and 0.4 on the scale at the side of the Garret Gas Train. Observe change in the colour of the dragger tube.
xii. Continue flowing gas for 15 minutes.
xiii. Record the maximum darkened length (in units marked on the tube) before the front starts to smear.
   A blank test has also been performed according to the above-detailed protocol, except that step x is absent (there is no injection of test Sample).
   The amount of sulphide in mg/l is calculated according to the following formula:

$$\frac{Darkened length on tube x Tube factor}{Volume}$$

### Wherein:

The tube facture is printed on dragger tube box,
The volume is the volume of the Test sample or of the blank, in ml
   The calculation is performed for the blank and for the test sample.
   The efficiency of the scavenger of the invention is detailed in table 6. The efficiency is expressed in percentage of the blank amount of sulphide.

TABLE 6
H2S scavenging efficiency

| Scavenger | BLANK | I1 |
|---|---|---|
| Treatment, in % wt | | 1% |
| Efficiency | 6000 mg/l | 50% |

As illustrated in Table 6, the scavenger product of the invention replying to formula (IIA) allows reducing the H₂S content in a drilling fluid of 50% wt. It can be noted that the reduction of the H₂S content can still be improved if the treat rate is increased, for example up to an amount of 2% wt.

1.-15. (canceled)

16. A composition comprising the reaction product of at least one phenol compound with aldehyde(s), wherein the phenol compound(s) reply to formula (II): 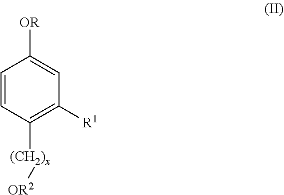
wherein
R represents a hydrogen atom or a hydrocarbyl group comprising from 1 to 30 carbon atoms,
x is 1 or 2,
R¹ is a linear alkyl or alkenyl group having 15 carbon atoms if x is 1,
R¹ is hydrogen if x is 2,
R² represents a hydrogen atom or a hydrocarbyl group comprising from 1 to 30 carbon atoms.

17. The composition according to claim 16, wherein the phenol compound is selected from tyrosol, 3-pentadecylphenol and cardanol.

18. The composition according to claim 16, wherein aldehydes are selected from formaldehydes and paraformaldehydes.

19. The composition according to claim 16, wherein the reaction product comprises at least one compound of formula (I): 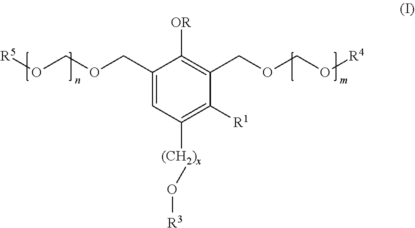
Wherein
R represents a hydrogen atom or a hydrocarbyl group comprising from 1 to 30 carbon atoms,
n and m are, independently to each other, an integer ranging from 0 to 6,
x is 1 or 2,
R¹ is a linear alkyl or alkenyl group having 15 carbon atoms if x is 1,
R¹ is hydrogen if x is 2,
R³ is identical to R² or R³ represents —(CH₂O)_{y}H or a linear or branched alkyl group having from 10 to 40 carbon atoms,
y is an integer ranging from 1 to 5,
R⁴ and R⁵ represent, independently to each other, a hydrogen atom or a hydrocarbyl group comprising from 1 to 30 carbon atoms.

20. The composition according to claim 19, wherein the compound of formula (I) is selected from compounds of formula (Ib): 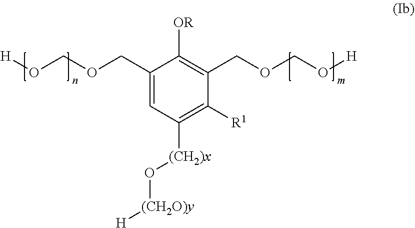
Wherein:
R is a hydrogen atom or a linear, branched, cyclic, alkyl or alkenyl group comprising from 1 to 16 carbon atoms,
R¹ is hydrogen,
x ranges from 2 to 4,
n and m are identical and ranges from 0 to 4,
y ranges from 1 to 4.

21. The composition according to claim 19, wherein R¹ is hydrogen.

22. Composition according to claim 20, wherein x ranges from 2 to 3.

23. The composition according to claim 19, wherein n and m are identical and range from 0 to 2.

24. The composition according to claim 19, wherein y ranges from 1 to 4.

25. The composition according to claim 16, comprising:
from 20 to 98% wt of the reaction product, and
from 2 to 80% wt of solvent(s),
based on the total weight of the composition.

26. A method for scavenging hydrogen sulphide and/or mercaptans in a stream, the method comprising contacting the streams with a scavenging composition,
wherein the stream is selected from hydrocarbon streams and water-containing streams and
wherein the scavenging composition comprises the reaction product of at least one phenol compound with aldehyde(s), wherein the phenol compound(s) reply to formula (II): 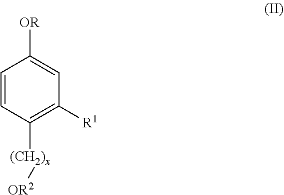
wherein
R represents a hydrogen atom or a hydrocarbyl group comprising from 1 to 30 carbon atoms,
x is 1 or 2,
R¹ is a linear alkyl or alkenyl group having 15 carbon atoms if x is 1,
R¹ is hydrogen if x is 2,
R² represents a hydrogen atom or a hydrocarbyl group comprising from 1 to 30 carbon atoms.

27. The method according to claim 26, wherein the reaction product is added in an amount of at least 1% wt, based on the total weight of hydrogen sulphide and mercaptans in the stream.

28. The method according to claim 26, wherein the hydrocarbon-containing streams are selected from liquefied petroleum gas (LPG), finished fuels, crude and heavy residual oils, natural gas asphalt, and oil-based drilling fluids.

29. A hydrocarbon-containing composition comprising hydrocarbons and the composition according to claim 16.

30. A water-containing stream comprising water and the composition according to claim 16.

31. The method according to claim 26, wherein the water-containing streams are selected from water-based drilling fluids.

32. The composition according to claim 19, wherein y ranges from 1 to 3.

33. Composition according to claim 20, wherein x is 2.

34. The composition according to claim 19, wherein n and m are identical and range from 0 to 1.

35. The composition according to claim 17, comprising:
from 20 to 98% wt of the reaction product, and
from 2 to 80% wt of solvent(s),
based on the total weight of the composition.
